(19) United States
(12) Patent Application Publication
(10) Pub. No.: US 2021/0106817 A1
(43) Pub. Date: Apr. 15, 2021

(54) MRI-SAFE IMPLANTABLE LEADS WITH HIGH-DIELECTRIC COATING

(71) Applicant: THE GENERAL HOSPITAL CORPORATION, BOSTON, MA (US)

(72) Inventors: Laleh Golestani Rad, Brookline, MA (US); Lawrence L. Wald, Cambridge, MA (US); Giorgio Bonmassar, Lexington, MA (US)

(21) Appl. No.: 16/970,550

(22) PCT Filed: Feb. 18, 2019
(86) PCT No.: PCT/US19/18399

§ 371 Date: Aug. 17, 2020

Related U.S. Application Data
(60) Provisional application No. 62/631,925, filed on Feb. 18, 2018.

(51) Int. Cl. A61N 1/08; H01B 7/02; H01B 7/00; A61N 1/05

(52) U.S. Cl. CPC A61N 1/086; A61N 1/05; H01B 7/0009; H01B 7/02

(57) ABSTRACT

A conductive lead apparatus for an implantable medical device includes a first conductor having a first outer diameter and a length, a high-dielectric constant layer having a second outer diameter and disposed around the first outer diameter of the first conductor, and a second conductor disposed around the second outer diameter of the high dielectric constant layer. The first conductor, high dielectric constant layer and the second conductor form a distributed capacitance along the length of the first conductor.

### CROSS-REFERENCE TO RELATED APPLICATIONS

This application is based on, claims priority to, and incorporates herein by reference in its entirety U.S. Ser. No. 62/631,925 filed Feb. 18, 2018, and entitled “System And Method For Reducing Heating of Implanted Leads Through High-Dielectric Capacitive Bleeding of Current (HD-CBLOC): Concepts, Simulations and Experimental Results.”

### BACKGROUND

The present disclosure relates generally to implantable medical devices and more particularly to an implantable conductive lead for use in conjunction with an implantable medical device where the conductive lead includes a high-dielectric constant layer to enable high-dielectric capacitive bleeding of currents to reduce heating generated by RF fields from an MRI system.

There has been a steady growth in the use of implantable electronic stimulation devices for therapeutic applications in the U.S. and globally. For example, the use of cardiac devices such as permanent pacemakers, cardioverter defibrillators, or cardiac resynchronization therapy devices and devices for neuromodulation such as for spinal cord stimulation (SCS), sacral nerve stimulation, and deep brain stimulation (DBS) continues to grow. Factors such as aging population, increasing prevalence of cardiovascular and neurological diseases, expanded target application, and new indications of use are among those driving such growth.

For a majority of neurologic, cardiac and musculoskeletal disorders, magnetic resonance imaging (MRI) is the diagnostic modality of choice because of its excellent soft tissue contrast and non-invasive nature. It is estimated that 50-75% of patients with cardiovascular disease may need to undergo MRI over their lifetime for non-cardiac or cardiac indications, with many patients requiring repeated examinations. Similarly, patients with neuromodulation devices such as DBS greatly benefit from MM exams, both for target verification and for post-operative monitoring of treatment-induced changes in the function of affected brain networks. Unfortunately, however, the interaction of radio frequency (RF) fields of MM transmitters with implanted leads results in safety hazards that severely limit the post-operative accessibility of MRI for patients with implanted conductive leads.

One major concern is RF-induced heating of tissue due to the “antenna effect” of the leads, wherein the electric field induced in the body couples with the elongated conductive leads and amplifies the specific absorption rate (SAR) of the RF energy in the tissue with respect to SAR values in the absence of conductive implants. Such a SAR amplification can cause excessive tissue heating and potential tissue damage. As a result, the conditions under which patients with implanted leads can receive an MRI are restrictive and often such patients are unable to receive an MRI.

Efforts to alleviate the problem of implant-induced tissue heating during MRI can be classified into three main categories: those that aim to modify the imaging hardware to make it less interactive with conducive implants, those that modify the implant structure and material to reduce the antenna effect, and those that, through surgical planning, modify the implant trajectory to reduce the coupling and the antenna effect. Examples of hardware modifications include the use of dual-drive birdcage coils to generate steerable low-E field regions that coincide with the implant, the introduction of rotating linear birdcage coils that allow individual patient adjustments for low SAR imaging, and parallel transmit systems that produce implant friendly modes. Alteration of the lead geometry includes techniques that aim to increase the lead's impedance to reduce the induced RF currents. Other recent examples suggest the use of resistive tampered stripline to scatter the RF energy along the length of the lead and reduce its concentration at the tip, use of external traps which couple to lead wires and take the RF energy away from internal wires, and the use of conductive pins to connect lead wires to the tissue and shunt inducted currents. Despite these efforts, however, the number of MR Safe or MR Conditional implantable leads remains limited.

Therefore, there is a need for conductive leads that improve RF safety of implantable electronic devices in MRI environment.

### SUMMARY

In accordance with an embodiment, a conductive lead apparatus for an implantable medical device includes a first conductor having a first outer diameter and a length, a high-dielectric constant layer having a second outer diameter and disposed around the first outer diameter of the first conductor, and a second conductor disposed around the second outer diameter of the high dielectric constant layer. The first conductor, high dielectric constant layer and the second conductor form a distributed capacitance along the length of the first conductor.

In accordance another embodiment, a conductive lead apparatus for an implantable medical device includes a first conductor having a first outer diameter and a length and a high-dielectric constant layer disposed around the first outer diameter of the first conductor, wherein the high-dielectric constant layer is configured to be in direct contact with a tissue of a subject. The first conductor and high dielectric constant layer form a distributed capacitance along the length of the first conductor.

### BRIEF DESCRIPTION OF THE DRAWINGS

The present invention will hereafter be described with reference to the accompanying drawings, wherein like reference numerals denote like elements.

FIG. 1 is a perspective view of a conductive lead in accordance with an embodiment;

FIG. 2 is a cross-sectional view of a conductive lead in accordance with an embodiment;

FIG. 3 is a circuit diagram corresponding to the conductive lead of FIGS. 1 and 2 in accordance with an embodiment;

FIG. 4 is a perspective view of a conductive lead in accordance with an embodiment;

FIG. 5 is a cross-sectional view of a conductive lead in accordance with an embodiment;

FIG. 6 is a circuit diagram corresponding to the conductive lead of FIGS. 4 and 5 in accordance with an embodiment; and

FIG. 7 is a graph illustrating the relative permittivity of a high-dielectric constant paste in accordance with an embodiment.

### DETAILED DESCRIPTION

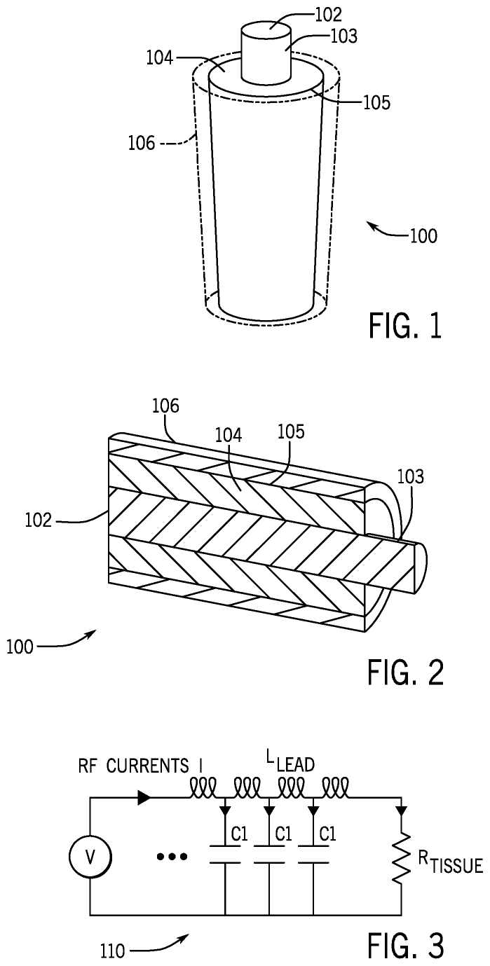

FIG. 1 is a perspective view of a conductive lead in accordance with an embodiment and

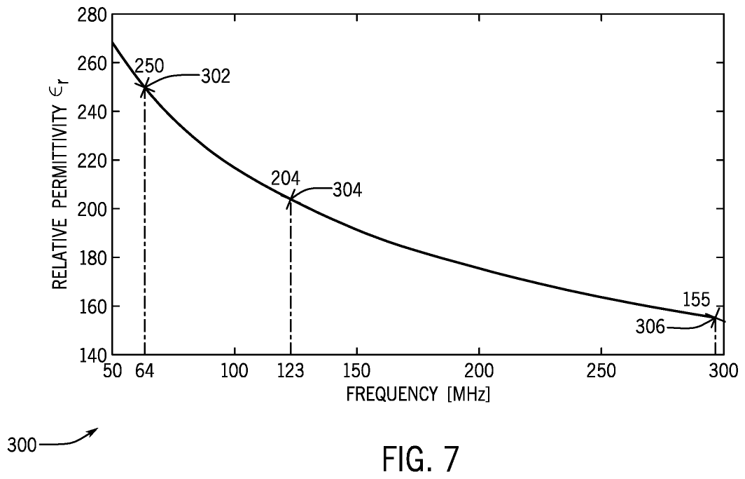

FIG. 2 is a cross-sectional view of a conductive lead in accordance with an embodiment. Conductive lead 100 may be used in an implantable stimulation device such as, for example, deep brain stimulation devices, spinal cord stimulation devices, cardiac pacemakers and cardiac defibrillators. For example, an end of conductive lead 100 may be attached to an electrode configured to provide therapy to a patient. As shown in FIGS. 1 and 3, conductive lead 100 includes a first conductor 102 that has an outer diameter 103. In one embodiment, the first conductor 102 includes one or more wires, for example, insulated copper wire. Conductor 100 also includes a high-dielectric constant (HDC) layer 104 that is disposed around the first outer diameter 103 of the first conductor 102 to form a high-permittivity insulation. The HDC layer 104 also has a second outer diameter 105. In an embodiment, the HDC layer 104 is a non-toxic HDC material in the form of a suspension that is used to create a thin layer (e.g., 1.2 mm) that coats the first conductor 102. In various embodiments, a high-permittivity powder may be used to form the suspension (e.g., a paste). For example, the HDC layer 104 may be a high permittivity (e.g., 100<εr<400) paste composed of Barium titanate (BaTiO3) suspended in distilled and de-ionized water. In this embodiment, a chemical dispersant (e.g., sodium polyacrylate) may also be added to the BaTiO2 suspension. The BaTiO3 suspension forms an ultra-high permittivity insulation as shown by FIG. 7. The permittivity 302 of a BaTiO3 suspension measured at 64 MHz (1.5 T) was εr=250. The permittivity 304 of a BaTiO3rf suspension measured at 123 MHz (3 T) was εr=204. The permittivity 306 of a BaTiO3 suspension measured at 279 MHz (7 T) was εr=155.

Returning to FIGS. 1 and 2, the conductive lead 100 also includes a second conductor 106 that is disposed around the second outer diameter 105 if the HDC layer 104. The second conductor 106 may be, for example, in the form of tubing. In one embodiment, the second conductor 106 is composed of a semi-conductive or weakly conductive material. For example, the second conductor 106 may be partially conductive carbon-doped silicon tubing that is non-magnetic and non-toxic.

FIG. 3 is a circuit diagram corresponding to the conductive lead of FIGS. 1 and 2 in accordance with an embodiment. Circuit 110 is an equivalent circuit model of the conductive lead 100 shown in FIGS. 1 and 2. The conductive lead 100 improves MRI RF safety through capacitive bleeding of currents along the length of the lead 100 which may be referred to as high-dielectric capacitive bleeding of current (HD-CBLOC). Referring to FIGS. 1-3, the HDC layer 104 and the second conductor 106 form a continuous capacitive element surrounding the first conductor 102. The first conductor 102, HDC layer 104 and second conductor 106 form a distributed capacitance along the length of the lead between the first conductor 102 and the conductive issue of the subject (not shown). The resulting circuit (e.g., equivalent circuit 110) dissipates RF energy through the length of the lead (e.g., the length of the first conductor 102) through the distributed capacitance in the form of displacement currents. Accordingly, the RF energy applied during an MM scan is dissipated before it reaches an end or tip of the lead (e.g., an exposed tip used for providing therapy to a subject) and thereby reduces the energy concentration at the tip of the lead. By shunting the RF energy along the length of the lead, the RF heating at the tip of the lead and heating of the tissue near the lead (e.g., the tip of the lead) during an Mill scan is significantly reduced. In one example, the temperature increase (heating) generated by the lead 200 during an Mill scan is less than 1° C. In addition, the specific absorption rate (SAR) near the tip of the lead is also significantly reduced. In one example, the SAR of tissue near the tip of the lead during an Mill scan is 8 W/kg. In an embodiment, metal artifacts are reduced or eliminated which may render the tip of the conductive lead 100 directly observable in both axial and sagittal MR images. In various embodiment, the conductive lead 100 reduces heating and SAR during Mill at 1.5 T, 3 T and 7 T.

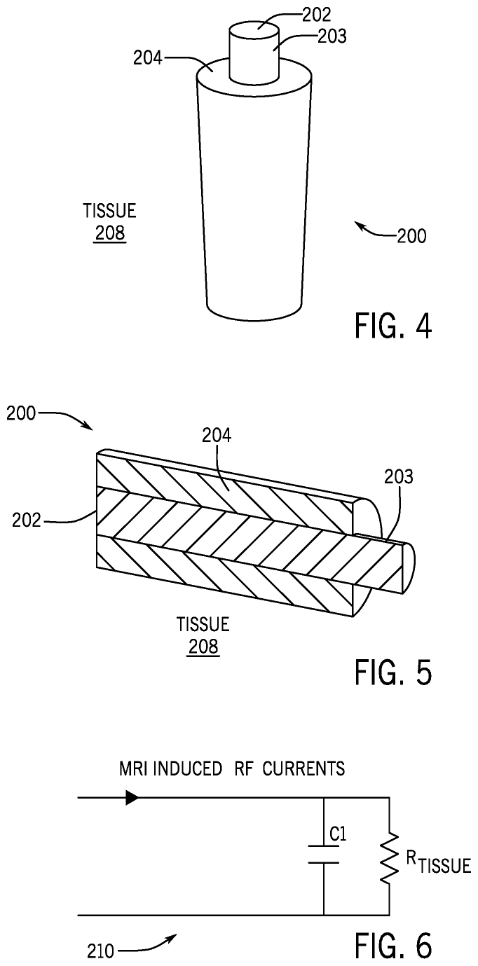

In another embodiment, the HDC layer may be in the form of a solid material that may be in direct contact with conductive tissue of a subject so as not to require the second conductor (e.g., conductive tubing). FIG. 4 is a perspective view of a conductive lead in accordance with an embodiment and FIG. 5 is a cross-sectional view of a conductive lead in accordance with an embodiment. Conductive lead 200 may be used in an implantable stimulation device such as, for example, deep brain stimulation devices, spinal cord stimulation devices, cardiac pacemakers and cardiac defibrillators. For example, an end of conductive lead 200 may be attached to an electrode configured to provide therapy to a patient. As shown in FIGS. 4 and 5, conductive lead 200 includes a first conductor 202 that has an outer diameter 203. In one embodiment, the first conductor 202 includes one or more wires, for example, insulated copper wire. Conductor 200 also includes a high-dielectric constant (HDC) layer 204 that is disposed around the first outer diameter 203 of the first conductor 202 to form a high-permittivity insulation. In an embodiment, the HDC layer 204 is a non-toxic solid HDC material that is configured to be in direct contact with the conductive tissue 208 of a subject. In one example, the solid HC layer is composed of AI203. In an embodiment, the solid HDC layer 204 is deposited on the first conductor using atomic layer deposition techniques for deposition of high-dielectric oxides. The solid HDC layer 204 may act as insulation for the first conductor 202.

FIG. 6 is a circuit diagram corresponding to the conductive lead of FIGS. 4 and 5 in accordance with an embodiment. Circuit 210 is an equivalent circuit model of the conductive lead 200 shown in FIGS. 4 and 5. The conductive lead 200 improves MRI RF safety through capacitive bleeding of currents along the length of the lead 200 which may be referred to as high-dielectric capacitive bleeding of current (HD-CBLOC). Referring to FIGS. 4-6, the HDC layer 204 forms a continuous capacitive element surrounding the first conductor 202. The first conductor 102 and HDC layer 204 form a distributed capacitance along the length of the lead between the first conductor 202 and the conductive tissue 208 of the subject. The resulting circuit (e.g., equivalent circuit 210) dissipates RF energy through the length of the lead (e.g., the length of the first conductor 202) through the distributed capacitance in the form of displacement currents. Accordingly, the RF energy applied during an MM scan is dissipated before it reaches an end or tip of the lead (e.g., an exposed tip used for providing therapy to a subject) and thereby reduces the energy concentration at the tip of the lead. By shunting the RF energy along the length of the lead, the RF heating at the tip of the lead and heating of the tissue near the lead (e.g., the tip of the lead) during an MRI scan is significantly reduced. In one example, the temperature increase (heating) generated by the lead 200 during an MM scan is less than 1° C. In addition, the specific absorption rate (SAR) of tissue near the tip of the lead is also significantly reduced. In one example, the SAR of tissue near the tip of the lead during an MRI scan is 8 W/kg. In an embodiment, metal artifacts are reduced or eliminated which may render the tip of the conductive lead 200 directly observable in both axial and sagittal MR images. In various embodiment, the conductive lead 200 reduces heating and SAR during MRI at 1.5 T, 3 T and 7 T.

The present invention has been described in terms of one or more preferred embodiments, and it should be appreciated that many equivalents, alternatives, variations, and modifications, aside from those expressly states, are possible and within the scope of the invention.

1. A conductive lead apparatus for an implantable medical device, the conductive lead apparatus comprising:
a first conductor having a first outer diameter and a length;
a high-dielectric constant layer having a second outer diameter and disposed around the first outer diameter of the first conductor; and
a second conductor disposed around the second outer diameter of the high dielectric constant layer;
wherein the first conductor, high dielectric constant layer and the second conductor form a distributed capacitance along the length of the first conductor.

2. The apparatus according to claim 1, wherein the first conductor includes at least one wire.

3. The apparatus according to claim 1, wherein the high-dielectric constant layer is a suspension.

4. The apparatus according to claim 3, wherein the suspension is a paste formed using a high-permittivity powder suspended in de-ionized water.

5. The apparatus according to claim 1, wherein the distributed capacitance dissipates RF energy through the length of the first conductor.

6. The apparatus according to claim 5, wherein the RF energy is dissipated in the form of displacement currents.

7. The apparatus according to claim 5, wherein the dissipation of RF energy results in a reduction of heating of a tip of the conductive lead.

8. The apparatus according to claim 5, wherein the dissipation of RF energy results in a reduction of specific absorption rate in a tissue of a subject near a tip of the conductive lead. WO 2019/161324 PCT/US2019/018399

9. The apparatus according to claim 5, wherein the dissipation of RF energy results in a reduction of metal artifacts in MRI images of a tip of the conductive lead.

10. The apparatus according to claim 1, wherein the second conductor is a conductive tubing.

11. The apparatus according to claim 10, wherein the conductive tubing is carbon-doped silicon tubing.

12. A conductive lead apparatus for an implantable medical device, the conductive lead apparatus comprising:
a first conductor having a first outer diameter and a length; and
a high-dielectric constant layer disposed around the first outer diameter of the first conductor, wherein the high-dielectric constant layer is configured to be in direct contact with a tissue of a subject;
wherein the first conductor and high dielectric constant layer form a distributed capacitance along the length of the first conductor.

13. The apparatus according to claim 12, wherein the high-dielectric constant layer is formed from a solid material.

14. The apparatus according to claim 13, wherein the high-dielectric layer is formed using Al₂O₃.

15. The apparatus according to claim 12, wherein the first conductor includes at least one wire.

16. The apparatus according to claim 12, wherein the distributed capacitance dissipates RF energy through the length of the first conductor.

17. The apparatus according to claim 16, wherein the RF energy is dissipated in the form of displacement currents.

18. The apparatus according to claim 16, wherein the dissipation of RF energy results in a reduction of heating of a tip of the conductive lead.

19. The apparatus according to claim 16, wherein the dissipation of RF energy results in a reduction of specific absorption rate in a tissue of a subject near a tip of the conductive lead.

20. The apparatus according to claim 16, wherein the dissipation of RF energy results in a reduction of metal artifacts in MRI images of a tip of the conductive lead.
